(19) United States
(12) Patent Application Publication
(10) Pub. No.: US 2022/0238217 A1
(43) Pub. Date: Jul. 28, 2022

(54) SCHEDULE MANAGEMENT APPARATUS, ULTRASONIC DIAGNOSTIC APPARATUS, AND NON-TRANSITORY COMPUTER-READABLE STORAGE MEDIUM STORING SCHEDULE MANAGEMENT PROGRAM

(71) Applicant: CANON MEDICAL SYSTEMS CORPORATION, Otawara-shi (JP)

(72) Inventor: Koichi MORIKAWA, Otawara (JP)

(73) Assignee: CANON MEDICAL SYSTEMS CORPORATION, Otawara-shi (JP)

(21) Appl. No.: 17/579,830

(22) Filed: Jan. 20, 2022

(30) Foreign Application Priority Data
Jan. 26, 2021 (JP) 2021-010685

(51) Int. Cl. G16H 40/40; G16H 40/20

(52) U.S. Cl. CPC G16H 40/40; G16H 40/20

(57) ABSTRACT

A schedule management apparatus of one embodiment is configured to be connectable to an ultrasonic diagnostic apparatus that is provided with at least one ultrasonic probe, and includes processing circuitry configured to: provisionally determine a first inspection schedule of the ultrasonic probe from inspection history information of the ultrasonic probe; acquire examination reservation information of an object; presume a usage schedule of the ultrasonic probe from the examination reservation information; and determine a second inspection schedule of the ultrasonic probe based on the first inspection schedule and the usage schedule, by adjusting the first inspection schedule in such a manner that usage time of the ultrasonic probe in the usage schedule does not overlap with inspection time of the ultrasonic probe in the first inspection schedule.

### CROSS-REFERENCE TO RELATED APPLICATION

This application is based upon and claims the benefit of priority from Japanese Patent Application No. 2021-010685, filed on Jan. 26, 2021, the entire contents of which are incorporated herein by reference.

### FIELD

Disclosed Embodiments relate to a schedule management apparatus, an ultrasonic diagnostic apparatus, and a non-transitory computer-readable storage medium storing a schedule management program.

### BACKGROUND

An ultrasonic diagnostic apparatus transmits an ultrasonic pulse and/or an ultrasonic continuous wave generated by transducers included in an ultrasonic probe into an object's body, and converts a reflected ultrasonic wave caused by difference in acoustic impedance between tissues inside the object into an electric signal by using the transducers to non-invasively acquire information inside the object. A medical examination using an ultrasonic diagnostic apparatus can readily generate and acquire medical images such as tomographic images and three-dimensional images inside an object by only bringing the ultrasonic probe into contact with the body surface, and thus, is widely applied to morphological diagnosis and functional diagnosis of an organ.

Since a normal operation of the ultrasonic probe is indispensable for making a correct diagnosis, an inspection of the ultrasonic probe is performed conventionally. Typical inspections of ultrasonic probes include periodic inspections, for example, every year or every two years. In recent years, there has been a movement to make regular inspections of ultrasonic probes mandatory by law or regulation.

During the inspection of an ultrasonic probe, the ultrasonic probe cannot be used for an examination of a patient. Thus, it is important to create an inspection schedule such that periodic inspections of the ultrasonic probes can be performed at the scheduled time, while securing usage time of the ultrasonic probes necessary for examinations.

The larger the medical institution is, the greater the number of ultrasonic probes used in the medical institution becomes. In addition, since the types of ultrasonic probes differ depending on what part of the patient's body is examined and how the examination is conducted, there are a wide variety of types of ultrasonic probes.

Thus, manually creating an inspection schedule of ultrasonic probes is a considerably complicated task. Furthermore, change in usage schedule of the ultrasonic probes may frequently occur, and it is not easy to flexibly change the inspection schedule in response to the change in the usage schedule of the ultrasonic probes.

Accordingly, there is a demand for a method and a system for readily generating and managing an inspection schedule of an ultrasonic probe.

### BRIEF DESCRIPTION OF THE DRAWINGS

In the accompanying drawings:

FIG. 1 is a perspective view illustrating an appearance of an ultrasonic diagnostic apparatus provided with ultrasonic probes to be managed by a schedule management apparatus according to the first embodiment;

FIG. 2 is a diagram illustrating a configuration of a probe inspection schedule management system in which a plurality of ultrasonic diagnostic apparatuses, a schedule management apparatus, and an examination reservation server are interconnected via a network;

FIG. 3 is a block diagram mainly illustrating a configuration of the schedule management apparatus;

FIG. 4 is a flowchart illustrating processing to be performed by the schedule management apparatus of the first embodiment;

FIG. 5 is a diagram illustrating a processing concept of an inspection history information acquisition function and a first inspection schedule determination function;

FIG. 6 is a diagram illustrating a processing concept of an examination reservation information acquisition function and a probe usage schedule presumption function;

FIG. 7 is a diagram illustrating a processing concept of a second inspection schedule determination function;

FIG. 8 is a diagram illustrating a processing concept of the second inspection schedule determination function according to the second embodiment; and

FIG. 9 is a diagram illustrating a processing concept of the second inspection schedule determination function according to the third embodiment.

### DETAILED DESCRIPTION

Hereinafter, embodiments of the present invention will be described by referring to the accompanying drawings.

A schedule management apparatus of one embodiment is configured to be connectable to an ultrasonic diagnostic apparatus that is provided with at least one ultrasonic probe, and includes processing circuitry configured to: provisionally determine a first inspection schedule of the ultrasonic probe from inspection history information of the ultrasonic probe; acquire examination reservation information of an object; presume a usage schedule of the ultrasonic probe from the examination reservation information; and determine a second inspection schedule of the ultrasonic probe based on the first inspection schedule and the usage schedule, by adjusting the first inspection schedule such that usage time of the ultrasonic probe does not overlap with its inspection time in the first inspection schedule.

### First Embodiment

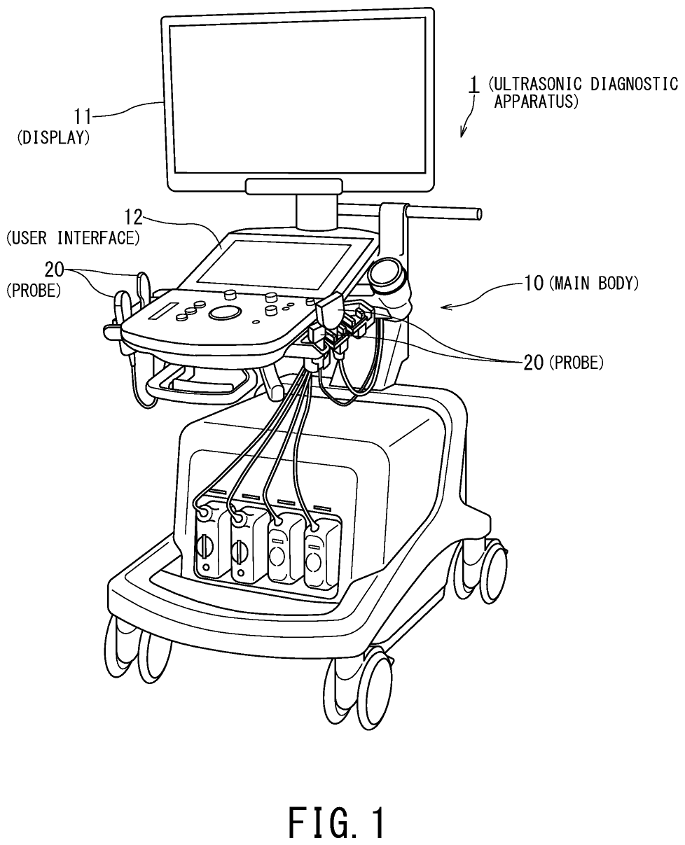

FIG. 1 is a perspective view illustrating an appearance of an ultrasonic diagnostic apparatus 1 provided with ultrasonic probes 20 (hereinafter, simply referred to as the probes 20), which is managed by a schedule management apparatus 100 according to the first Embodiment.

A main body 10 of the ultrasonic diagnostic apparatus 1 includes a display 11, a user interface 12, and various circuits housed in a body casing that is provided with casters.

The display 11 displays ultrasonic images and various data generated by the various circuits of the main body 10. The display 11 includes, for example, a liquid crystal display panel and/or an organic EL (Electro Luminescence) panel.

The user interface 12 is a device via which a user inputs various data and information into the main body 10 or sets various operation modes to the main body 10.

The ultrasonic diagnostic apparatus 1 includes a plurality of probes 20, for example, four probes 20 as shown in FIG. 1. Each probe 20 is configured to be attachable and detachable to/from the main body 10.

As described above, conventionally, the probes 20 are inspected in order to maintain the quality. Typical inspections of the probes 20 include a periodic inspection that is performed on a regular basis, for example, every year or every two years.

One or more probes 20 are inspected while being connected to the main body 10. Although the inspection method for the probes 20 is not limited to a specific inspection manner, the probes 20 can be inspected by, for example, measuring reception sensitivity of transducers included in each probe 20. The transducers may be referred to as vibrating elements or piezoelectric vibratos. In this inspection method, first, the probe 20 to be inspected is selected by the user via the user interface 12. Next, for example, the user inputs an instruction to start the inspection through the user interface 12.

In response to the instruction to start the inspection, for example, a transmission pulse for inspection is inputted from the transmission circuit of the main body 10 to the probe 20, and an ultrasonic signal for inspection is radiated from the probe 20 into the space. This radiated signal leaks into each transducer of the probe 20, and this leaked signal serves as an ultrasonic input signal for inspection of each transducer. The electric signal for this ultrasonic input signal is inputted as a channel signal from each transducer of the probe 20 to an image generation circuit of the main body 10.

The image generation circuit can measure the sensitivity of each transducer by, for example, measuring the magnitude of this channel signal. Whether or not there is an abnormality in the probe 20 can be determined from the results of the sensitivity measurement of each transducer. The inspection results such as presence/absence of an abnormality in the probe 20 are displayed on the touch panel of the user interface 12, for example.

In addition to the above-described method, in another conceivable inspection method, a phantom for inspection is imaged by using the probe 20 to be inspected and the user observes the obtained image to determine whether the probe 20 is normal or abnormal, for example.

The inspection result is stored as inspection history information in an appropriate memory inside the ultrasonic diagnostic apparatus 1 in addition to being displayed on the user interface 12. The inspection history information includes at least identification information of the probe 20 and information about when the inspection was conducted, such as the inspection date and time.

The identification information of the probe 20 includes its model name indicating the type of probe 20 and a serial number (sometimes called a manufacturing number) assigned specifically for each probe 20 (i.e., individual identification number for each probe). Such serial number and the model name of each probe 20 is stored in a non-volatile memory in the probe 20, for example. When the probe 20 is connected to the main body 10, its probe model name and serial number are read out from the memory in this probe 20 to the main body 10.

Further, when the above-described inspection of the probe 20 is performed, the model name and serial number of the probe 20 as well as the inspection date and time are stored as inspection history information in an appropriate memory inside the ultrasonic diagnostic apparatus 1.

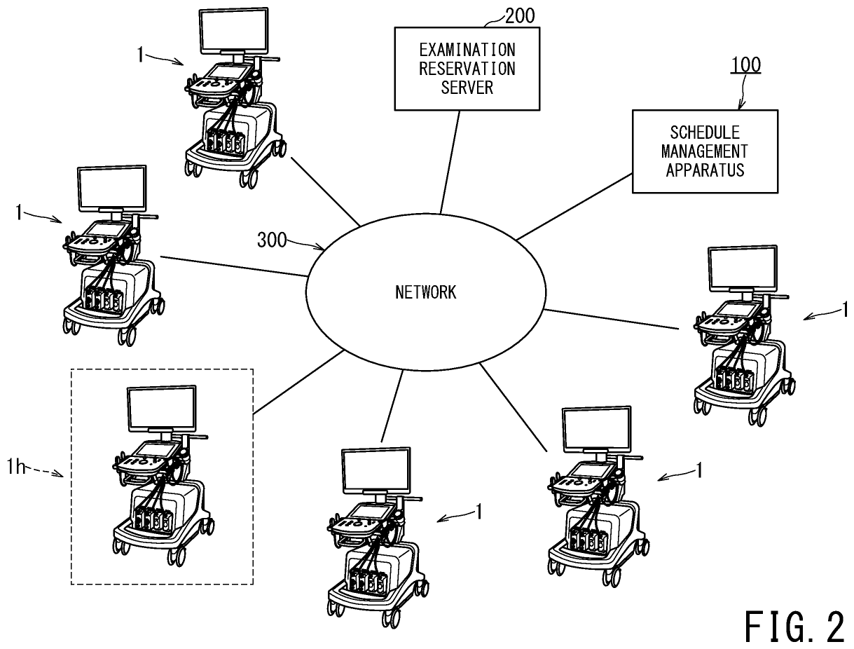

FIG. 2 is a diagram illustrating a configuration of a probe inspection schedule management system, in which a plurality of the ultrasonic diagnostic apparatuses 1, the schedule management apparatus 100, and an examination reservation server 200 are interconnected via a network.

Each of the plurality of ultrasonic diagnostic apparatuses 1 is used for inspecting the probes 20. The above-described inspection history information is sent from each ultrasonic diagnostic apparatus 1 to the schedule management apparatus 100 via the network 300. With such a configuration, the schedule management apparatus 100 can manage the inspection history information of each ultrasonic diagnostic apparatus 1 in an integrated manner. In other words, with such a configuration, the inspection history information of each ultrasonic diagnostic apparatus 1 is centrally managed by the schedule management apparatus 100. Details of the configuration and operation of the schedule management apparatus 100 will be described below.

The examination reservation server 200 is an information processing apparatus that manages examination reservations of patients. When a doctor examines a patient and determines that additional examination of the patient is necessary, the doctor reserves the examination of the patient. Although there are various types of examinations, here, an examination using the ultrasonic diagnostic apparatus 1 is assumed. The examination reservation information includes identification information of the patient to be examined, information indicating the outline and purpose of the examination, such as an abdominal examination or a circulatory organ examination, and information on the body part and/or examination organ/tissue to be examined using the probe 20, such as the liver, the heart, or legs.

Although the schedule management apparatus 100 and the examination reservation server 200 are described as separate configurations in FIG. 2, both can be combined into one configuration. For example, the function of the examination reservation server 200 can be included in the schedule management apparatus 100.

Further, the function of the schedule management apparatus 100 can be achieved by one of the plurality of ultrasonic diagnostic apparatuses 1, for example, by the ultrasonic diagnostic apparatus 1h serving as the host as shown in FIG. 2.

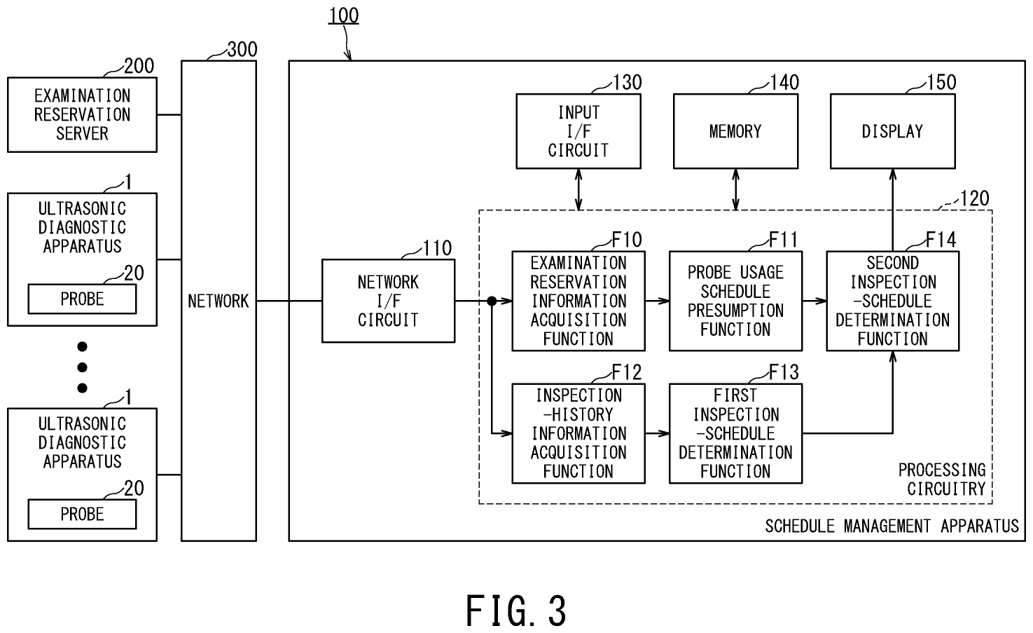

FIG. 3 is a block diagram illustrating a configuration of the probe inspection schedule management system illustrated in FIG. 2. In particular, the block diagram of in FIG. 3 illustrates the configuration of the schedule management apparatus 100 in detail.

As described above, the network 300 is connected to the examination reservation server 200 and the schedule management apparatus 100 in addition to the plurality of ultrasonic diagnostic apparatus 1. Although each ultrasonic diagnostic apparatus 1 is shown to have one probe 20 in FIG. 3, the number of probes 20 is not limited to one. A plurality of probes 20, for example, four probes 20 as shown in FIG. 1, can be attached to each ultrasonic diagnostic apparatus 1.

The schedule management apparatus 100 includes: a network I/F (interface) circuit 110 configured to exchange data via the network 300; processing circuitry 120; an input I/F (interface) circuit 130; a memory 140; and a display 150. The schedule management apparatus 100 is an information processing apparatus such as a personal computer or a workstation.

The input I/F circuit 130 includes various devices for an operator to input various data and information, such as a mouse, a keyboard, a trackball, and a touch panel. The input I/F circuit 130 also includes electronic circuits for exchanging signals between devices and the processing circuitry 120.

The memory 140 is a recording medium including a read-only memory (ROM) or a random access memory (RAM) in addition to an external memory device such as a hard disk drive (HDD) or an optical disc device. The memory 140 stores various data and information as well as various programs to be executed by the processor included in the processing circuitry 120.

The display 150 is a display device such as a liquid crystal display panel, a plasma display panel, and an organic EL panel.

The processing circuitry 120 is a circuit that includes a central processing unit (CPU) and/or a special-purpose or general-purpose processor, for example. The processor implements various functions described below by executing programs stored in the memory 140. The processing circuitry 120 may be configured as hardware such as a field programmable gate array (FPGA) and/or an application specific integrated circuit (ASIC). The various functions described below can also be implemented by such hardware. Or, the processing circuitry 120 may implement the various functions by combining hardware processing and software processing based on its processor and programs.

As shown in FIG. 3, the schedule management apparatus 100 causes the processing circuitry 120 to implement an examination reservation information acquisition function F10, a probe usage schedule presumption function F11, an inspection history information acquisition function F12, a first inspection schedule determination function F13, and a second inspection schedule determination function F14.

The inspection history information acquisition function F12 acquires the inspection history information of the probes 20 from each ultrasonic diagnostic apparatus 1, for example. The first inspection schedule determination function F13 provisionally determines an inspection schedule of the probes 20 as a first inspection schedule from the acquired inspection history information.

The examination reservation information acquisition function F10 acquires examination reservation information of objects (for example, one or more patients) from, for example, the examination reservation server 200. The probe usage schedule presumption function F11 presumes the usage schedule of the probes 20 from the acquired examination reservation information.

The second inspection schedule determination function F14 adjusts the first inspection schedule on the basis of the inspection schedule and the usage schedule of the probes 20 such that the usage time (i.e., time of use or when to use) indicated in the usage schedule of each probe 20 does note partially or entirely overlap with its inspection time indicated in the inspection schedule, and then determines the second inspection schedule of the probes 20 in accordance with the adjusted first inspection schedule.

The determined second inspection schedule is displayed, for example, on the display 150 so as to be notified to the user in charge of the inspection of the probes 20. Further, the determined second inspection schedule may be distributed to each of the plurality of ultrasonic diagnostic apparatuses 1 via the network 300. This distribution enables the user of the ultrasonic diagnostic apparatuses 1, such as a doctor or a medical imaging technologist, to be properly notified of which probe 20 to be inspected and the expected timing of inspection.

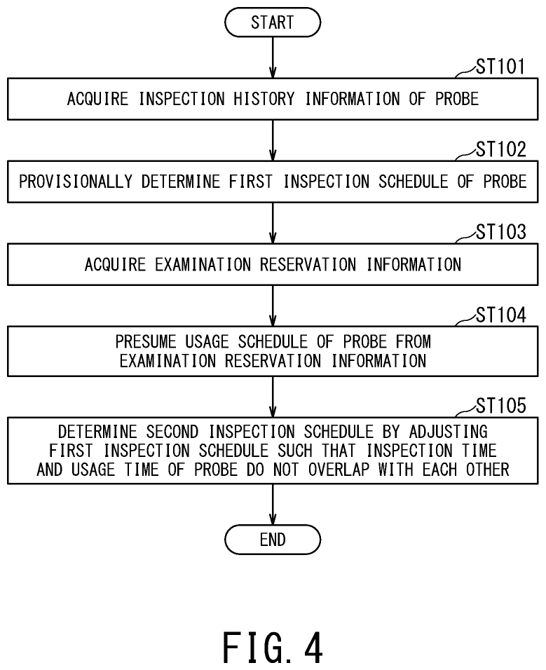

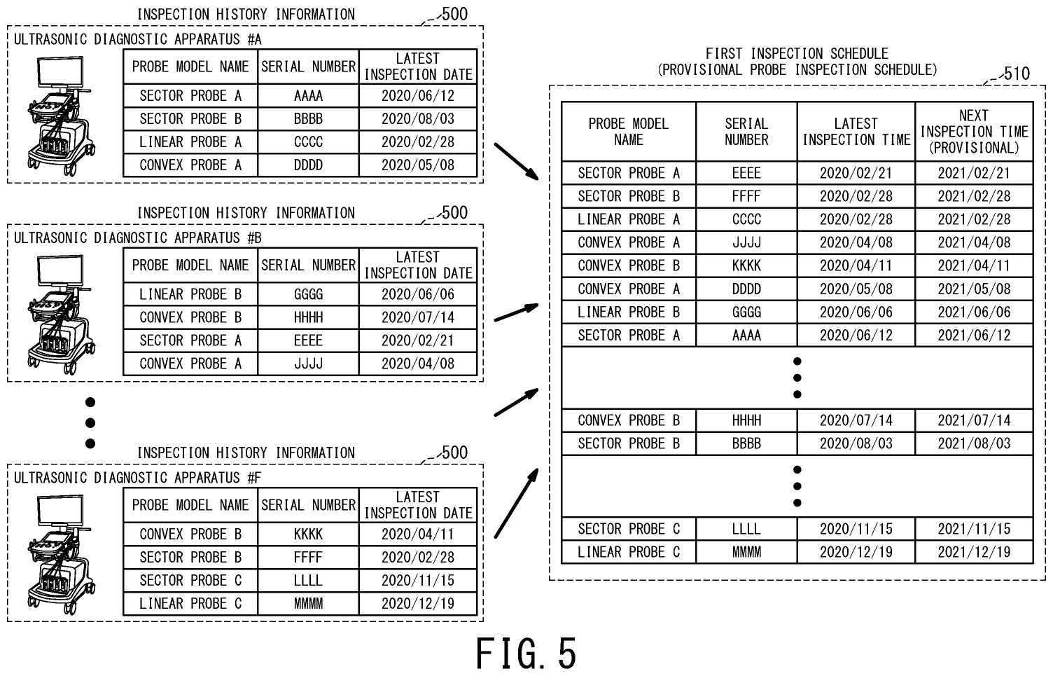

FIG. 4 is a flowchart illustrating the processing performed by the schedule management apparatus 100. Hereinafter, each of the above-described functions implemented by the processing circuitry 120 will be described in more detail by using the flowchart of FIG. 4 and the operation diagrams shown in FIG. 5 to FIG. 9.

First, in the step ST101 of FIG. 4, the inspection history information acquisition function F12 acquires the inspection history information of the probes 20.

In the next step ST102, the first inspection schedule determination function F13 provisionally determines the first inspection schedule of the probe 20.

FIG. 5 is a diagram illustrating the processing concept of the steps ST101 and ST102. The left side of FIG. 5 illustrates the inspection history information 500 generated and held in the plurality of ultrasonic diagnostic apparatuses 1 (for example, the ultrasonic diagnostic apparatuses #A to #F). As described above, the inspection history information 500 includes at least information on: the model name indicating the type of probe 20; the serial number indicating the individual identification number of the probe 20; and the latest inspection date of the probe 20 indicating the most recent date of the executed inspection.

Each ultrasonic diagnostic apparatus 1 stores the inspection history information 500 of its probes 20 in its own memory. For example, the ultrasonic diagnostic apparatus #A holds the inspection history information 500 indicating that the latest inspection date of the four probes, the sector probe A (serial number AAAA), the sector probe B (serial number BBBB), the linear probe A (serial number CCCC), and the convex probe A (serial number DDDD), are Jun. 12, 2020, Aug. 3, 2020, Feb. 28, 2020, and May 8, 2020, respectively.

The inspection history information 500 held by each ultrasonic diagnostic apparatus 1 is acquired by the inspection history information acquisition function F12 via the network 300. The first inspection schedule determination function F13 provisionally determines the inspection schedule of the probes 20 as the first inspection schedule from the acquired inspection history information 500.

The table on the right side of FIG. 5 illustrates the first inspection schedule 510. The first inspection schedule determination function F13 collects the inspection history information 500 acquired from the respective ultrasonic diagnostic apparatuses 1, and rearranges the probes 20, by referring to their ancillary information such as serial numbers according to the latest inspection time (or, the latest inspection date) in an order of an earlier time or date. Afterward, the first inspection schedule determination function F13 provisionally determines the date and time of the next inspection, based on the latest inspection time.

Note that it is “provisionally” determined since the “next inspection time” determined in the first inspection schedule 510 is not a final determination and may be adjusted depending on the usage status of the probes 20 as described below.

Although the method for determining the next inspection time is not limited to a specific method, the next inspection time can be determined from the periodic inspection interval and the latest inspection date (i.e., latest inspection time) by assuming that each probe 20 is inspected regularly, for example. The interval between regular inspections may be, for example, one year or two years. In the case of the first inspection schedule 510 shown in FIG. 5, the interval of the periodic inspection is assumed to be one year. Thus, the next inspection time for each probe 20 is determined by adding one year to each latest inspection date.

For example, in the first row of the table of the first inspection schedule 510, the latest inspection time of the sector probe A (with the serial number EEEE) is Feb. 21, 2020, so the next inspection time for this probe 20 is determined to be Feb. 21, 2021, which is one year after the latest inspection time. Similarly, in the second row of the table of the first inspection schedule 510, the latest inspection time of the sector probe B (with the serial number FFFF) is Feb. 28, 2020, so the next inspection time of this probe 20 is determined to be Feb. 28, 2021, which is one year after the latest inspection time.

In this manner, in the step ST102 of FIG. 4, the first inspection schedule of the probes 20 is provisionally determined.

Returning to FIG. 4, in the step ST103, the examination reservation information acquisition function F10 acquires the examination reservation information from, for example, the examination reservation server 200.

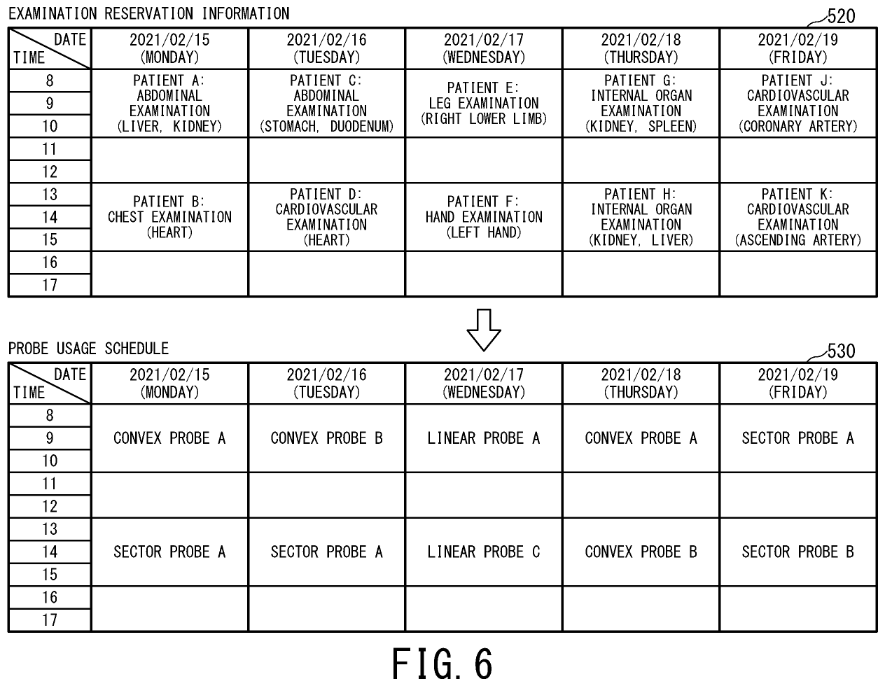

In the next step ST104, the probe usage schedule presumption function F11 presumes the usage schedule of the probe 20 from the acquired examination reservation information. FIG. 6 illustrates the processing concept of the steps ST103 and ST104.

The upper part of FIG. 6 is a table illustrating the examination reservation information 520. The examination reservation information 520 may be set by a user such as a doctor by using an examination reservation system (not shown), and the examination reservation information 520 is stored in the examination reservation server 200, for example.

The examination reservation information 520 includes identification information of the patient to be examined, the examination date, the time zone of the examination (or the start time), and may further include, for example, the purpose of the examination such as an abdominal examination or a cardiovascular examination, and the name of an organ and/or tissue, or a body part to be examined such as the liver, the kidney, and a coronary artery.

The lower part of FIG. 6 is a table illustrating the probe usage schedule 530 presumed from the examination reservation information 520. The probe usage schedule presumption function F11 refers to the examination reservation information 520 so as to presume the type of probe (or the model name corresponding to the probe type) to be used in the examination based on the purpose of the examination and information on the organ/tissue or body part to be examined, which are included in the examination reservation information 520.

For example, a probe type applicable to convex scanning is often used in an abdominal examination. Accordingly, a probe type applicable to convex scanning and a probe with a model name corresponding to this probe type can be presumed as a probe to be used for the abdominal examination. For example, in the (abdominal) examination of the time zone from 8:00 to 10:00 on Feb. 15 (Monday), 2021, a probe with the model name of “convex probe A” applicable to convex scanning can be presumed as the probe to be used in this time zone.

Further, in the examination of the circulatory system such as the heart and a coronary artery, a probe type applicable to sector scanning is often used. Accordingly, a probe type applicable to sector scanning and a probe with the model name corresponding to this probe type can be presumed as a probe to be used for the circulatory system examination. For example, in the (chest) examination of the time zone from 13:00 to 15:00 on Feb. 15 (Monday), 2021 and the (cardiovascular) examination of the time zone from 8:00 to 10:00 on Feb. 19 (Friday), 2021, the probe with the model name of “sector probe A” applicable to sector scanning can be presumed as the probe to be used for these time zones.

In addition, in leg examinations such as the right lower limb and finger examinations (i.e., hand examination), there are different types of probes suitable for each examination, so the probe with the model name suitable for such examinations can be presumed as a probe to be used for the examinations during the time zones from 8:00 to 10:00 and from 13:00 to 15:00 on Feb. 17 (Wednesday), 2021, for example.

In this manner, in the step ST104 of FIG. 4, the probe usage schedule 530 is presumed based on the examination reservation information 520.

It is also possible that a user, such as a doctor or a medical imaging technologist, include the type of the probe 20 and/or the model name of the probe 20 to be used for the corresponding examination in the examination reservation information 520. In this case, the type and/or the model name of the probe 20 to be used for each examination can be directly extracted from the examination reservation information 520, and the probe usage schedule 530 can be generated based on the extracted model name of the probe 20. Additionally or alternatively, the examination reservation information 520 may include the type and/or model name of the probe 20 which has been used in the past examinations for the same patient or in the similar examinations in the past.

Returning to FIG. 4, in the step ST105, the second inspection schedule determination function F14 adjusts the first inspection schedule 510 in such a manner that, for each probe 20, its usage time in the probe usage schedule 530 does not overlap with its inspection time in the first inspection schedule 510. The second inspection schedule determination function F14 determines the second inspection schedule 540 in accordance with the adjusted first inspection schedule 510.

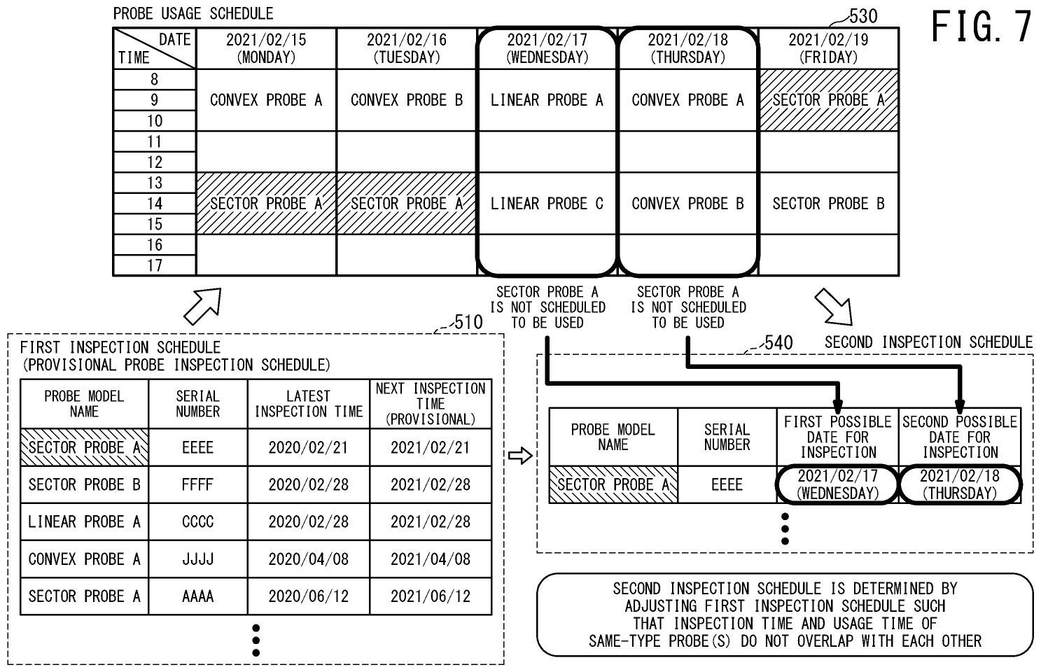

FIG. 7 illustrates the processing concept of the step ST105. The table on the lower left of FIG. 7 illustrates the first inspection schedule 510 and is a cutout of part of the table on the right side of FIG. 5. The table in the upper part of FIG. 7 is a table, in which hatchings and thick frames are added to the probe usage schedule 530 shown in the lower part of FIG. 6. The lower right of FIG. 7 illustrates the second inspection schedule 540 determined by the processing of the step ST105. In FIG. 7, hatchings and thick frames are added for illustrating the processing of the step ST105 of the first embodiment.

For example, in the step ST105, for the same type of the probe 20, the first inspection schedule is adjusted such that the usage time (i.e., when to use it) indicated in the usage schedule 530 does not overlap with the inspection time (i.e., when to inspect it) indicated in the provisionally determined first inspection schedule 510. The second inspection schedule 540 is determined in accordance with the adjusted first inspection schedule 510. In other words, the second inspection schedule 540 is determined by adjusting the first inspection schedule such that the inspection time and the usage time of the same type of the probe(s) do not overlap with each other.

For example, the first inspection schedule 510 indicates that the next inspection time for the probe with the model name of sector probe A (serial number EEEE) is Feb. 21, 2021. Such next inspection time refers to the deadline for the next inspection. Thus, the next inspection time means that the next inspection of the probe with the serial number EEEE should be executed by Feb. 21, 2021. In other words, the next inspection of the probe with the serial number EEEE should be executed on the deadline or within a specified number of days from the deadline of Feb. 21, 2021.

Meanwhile, the probe usage schedule 530 indicates that, during the one-week period (weekdays) before Feb. 21, 2021, a probe with the same model name of sector probe A as the probe with the serial number EEEE is scheduled to be used on Feb. 15, 2021 (Monday), Feb. 16 (Tuesday), and Feb. 19 (Friday).

Accordingly, in the step ST105, the second inspection schedule determination function F14 determines Feb. 17 (Wednesday) and Feb. 18 (Thursday), 2021 as possible dates for the next inspection date of the probe with the model name of sector probe A and the serial number EEEE, because Feb. 17 (Wednesday) and Feb. 18 (Thursday), 2021 are before the deadline of Feb. 21, 2021, and probe(s) with the model name of sector probe A is not scheduled to be used on these days.

For example, Feb. 17 (Wednesday), 2021, which is the earlier date, is determined as the first possible date for the inspection, and Feb. 18 (Thursday), 2021, which is the later date, is determined as the second possible date for the inspection. In this manner, in the step of the ST105, the first inspection schedule 510 is adjusted to determine the second inspection schedule 540.

### Second Embodiment

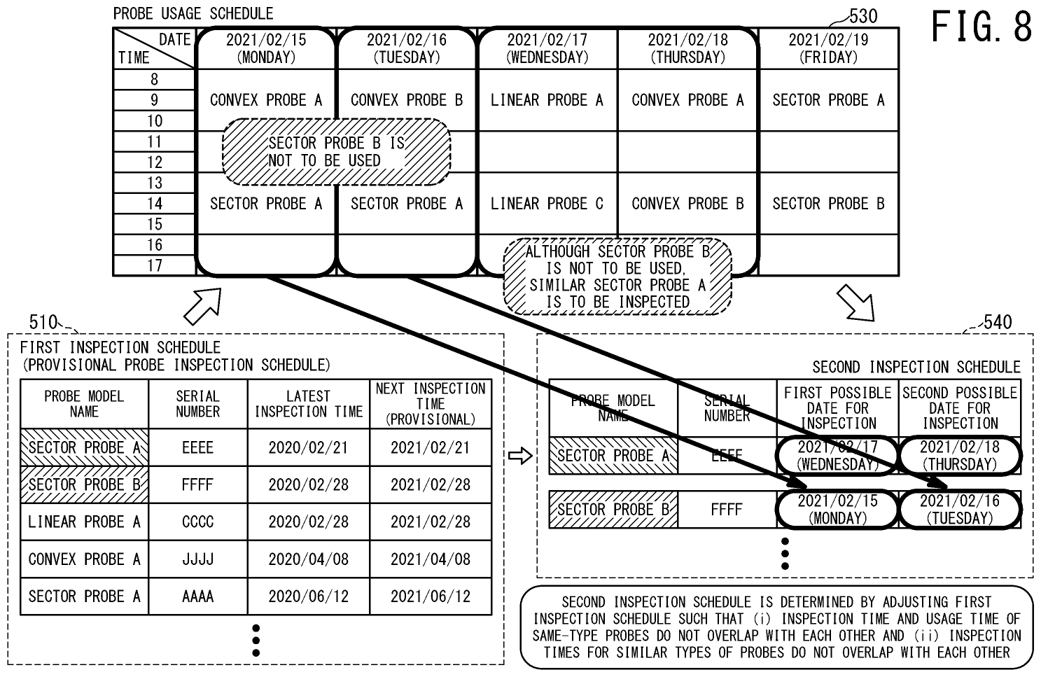

FIG. 8 is a diagram illustrating the processing concept of the step ST105 (i.e., second inspection schedule determination function) in the schedule management apparatus 100 according to the second embodiment. The only difference between the second embodiment and the first embodiment is the processing of the step ST105. The configuration of the second embodiment determines the second inspection schedule 540 by adjusting the first inspection schedule 510 such that, for the same type of probe 20, the usage time indicated in the usage schedule 530 and the inspection time indicated in the provisionally determined first inspection schedule 510 do not overlap with each other, and further, inspection times of similar types of probes do not overlap with each other.

In other words, the configuration of the second embodiment determines the second inspection schedule 540 by adjusting the first inspection schedule 510 such that (i) the usage time and the inspection time of the same type of probe do not overlap with each other, and (ii) inspection times of the similar types of probes do not overlap with each other.

An examination using one type of probe can often be performed by using a probe of similar type. Since the type of probe scheduled to be used for inspection may be broken and become unusable, and/or the number of inspections scheduled on the same inspection date may increase, there may be changes in inspection reservations. In such a case, if the same type of probe and a similar type of probe are inspected at the same date or in same time zone, it may be difficult to flexibly respond to the changes in the inspection reservation.

Thus, in the second embodiment, the second inspection schedule is determined by adjusting the first inspection schedule such that the inspection times of not only the same type of probe but also similar type of probe do not overlap with each other.

For example, FIG. 8 illustrates a case of determining the inspection time of the probe with the model name of sector probe B and serial number FFFF, which is similar to the probe with the model name of sector probe A. The probe with the model name of sector probe B is scheduled to be used on Feb. 17 (Wednesday) and Feb. 18 (Thursday), 2021. On the other hand, as shown in the second inspection schedule 540, for the probe with the model name of sector probe A, which is similar to the probe with the model name of sector probe B, Feb. 17 (Wednesday), 2021 has been determined as the first possible inspection date and Feb. 18 (Thursday), 2021 as the second possible inspection date. Also, on Feb. 15th (Monday) and Feb. 16th (Tuesday) in 2021, none of the probe with the model name of sector probe B, same model name as the one with the serial number FFFF, is scheduled to be used, and none of the probe with the model name of sector probe A, a model similar to the sector probe B is scheduled to be inspected. Thus, in the second embodiment, Feb. 15th (Monday) and Feb. 16th (Tuesday), 2021 are respectively determined as the first and the second possible inspection date.

### Third Embodiment

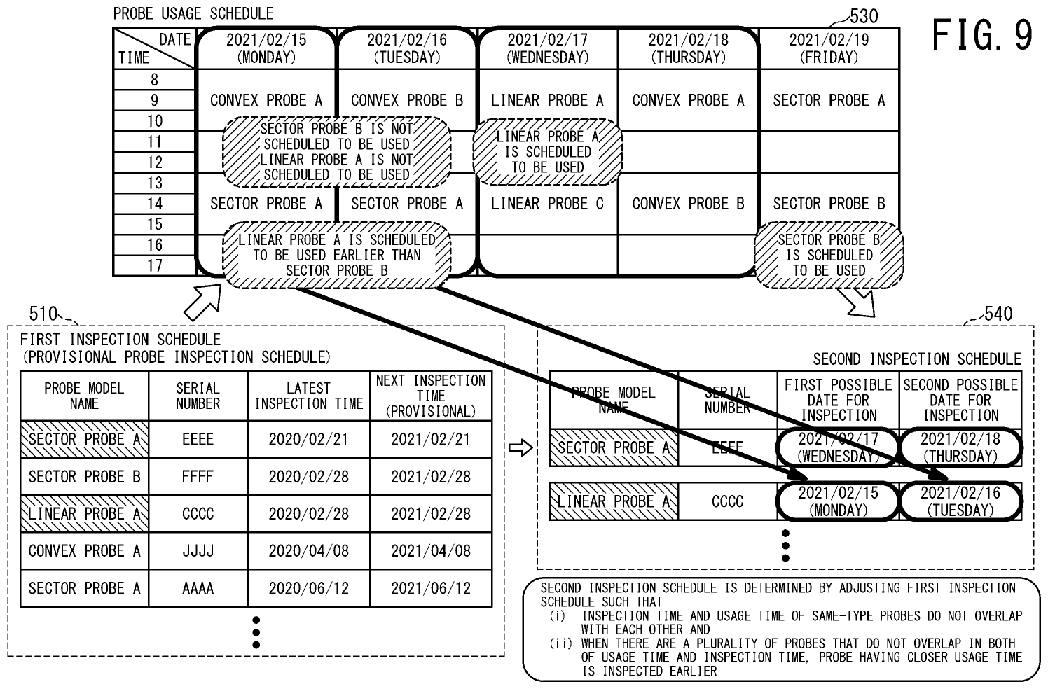

FIG. 9 illustrates the processing concept of the step ST105 (i.e., second inspection schedule determination function) in the schedule management apparatus 100 according to the third embodiment. The third embodiment differs from the first and second embodiments only in the processing of the step ST105.

The configuration of the third embodiment determines the second inspection schedule 540 by adjusting the first inspection schedule 510 such that the following first and second conditions are satisfied. Regarding the first condition, for the same type of probe 20, the usage time (i.e., when to use it) indicated in the usage schedule 530 and the inspection time (i.e., when to inspect it) indicated in the provisionally determined first inspection schedule 510 do not overlap with each other. In other words, the usage time and the inspection time of the same type of probe do not overlap with each other. Regarding the second condition, when there are a plurality of probes that do not overlap in both of the usage time and the inspection time, a probe having closer usage time is inspected earlier.

For example, as shown in FIG. 9, in the case of determining the inspection time of the probe with the model name of linear probe A and serial number CCCC, neither the probe with the model name of linear probe A nor the probe with the model name of sector probe B is scheduled to be used on Feb. 15th (Monday) and Feb. 16th (Tuesday), 2021. Also, the scheduled usage date of the probe with the model name of sector probe B is Feb. 19 (Friday), 2021, while the scheduled usage date of the probe with the model name of linear probe A is Feb. 17 (Tuesday), 2021. That is, the scheduled usage date of the probe with the model name of linear probe A comes earlier. Thus, in the third embodiment, the inspection time of the linear probe A (with serial number CCCC) is determined to be Feb. 15 (Monday), 2021 as the first possible inspection date, and Feb. 16 (Tuesday), 2021 as the second possible inspection date.

### Fourth Embodiment

As described above, the examination reservation information may often be changed depending on the convenience of the patient to be examined, the convenience of doctors, or the condition of the examination equipment. Thus, the schedule management apparatus 100 according to the fourth embodiment is configured to update the second inspection schedule each time the usage schedule of the ultrasonic probe is changed along with change in examination reservation information. For example, each time the examination reservation information 520 is acquired in the step ST10, the probe usage schedule presumption function F11 of the fourth embodiment monitors whether the examination reservation information 520 is changed or not. When it is determined that the examination reservation information 520 is changed, the probe usage schedule 530 is updated depending on the change. Further, when the examination reservation information 520 is changed, the second inspection schedule determination function F14 of the fourth embodiment updates the second inspection schedule 540 in response to the change, resulting that the inspection time of the probe 20 may be earlier or delayed.

According to the fourth embodiment, the inspection schedule management of the probes can be flexibly performed depending on change in the examination reservation.

According to the embodiments of the schedule management apparatus, the ultrasonic diagnostic apparatus, and the non-transitory computer-readable storage medium storing a schedule management program as described above, inspection schedules for ultrasonic probes used in the ultrasonic diagnostic apparatuses can be readily generated and be efficiently managed.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A schedule management apparatus that is connectable to an ultrasonic diagnostic apparatus provided with at least one ultrasonic probe, the schedule management apparatus comprising processing circuitry configured to:
provisionally determine a first inspection schedule of the ultrasonic probe from inspection history information of the ultrasonic probe;
acquire examination reservation information on an object;
presume a usage schedule of the ultrasonic probe from the examination reservation information; and
determine a second inspection schedule of the ultrasonic probe based on the first inspection schedule and the usage schedule, by adjusting the first inspection schedule in such a manner that usage time of the ultrasonic probe in the usage schedule does not overlap with inspection time of the ultrasonic probe in the first inspection schedule.

2. The schedule management apparatus according to claim 1, wherein the processing circuitry is configured to provisionally determine when the ultrasonic probe should be inspected next, as the first inspection schedule, based on a latest inspection time of the ultrasonic probe and a periodic inspection interval of the ultrasonic probe.

3. The schedule management apparatus according to claim 1, wherein:
the examination reservation information includes information on a scheduled examination date of the object, and at least one of information on a purpose of an examination and a body part of the object to be examined on the scheduled examination date; and
the processing circuitry is configured to presume the usage schedule of the ultrasonic probe from the information on the scheduled examination date of the object, and the at least one of the information on the purpose of the examination and the body part of the object to be examined on the scheduled examination date.

4. The schedule management apparatus according to claim 1, wherein:
the examination reservation information includes information on a scheduled examination date of the object, and at least one of information on a type of the ultrasonic probe to be used in an examination of the object on the scheduled examination date and a type of the ultrasonic probe that was used in a past examination of the object; and
the processing circuitry is configured to presume the usage schedule of the ultrasonic probe from the information on the scheduled examination date of the object, and the at least one of the information on the type of the ultrasonic probe to be used in the examination of the object on the scheduled examination date and the type of the ultrasonic probe that was used in the past examination of the object.

5. The schedule management apparatus according to claim 1, wherein:
the at least one ultrasonic probe comprises a plurality of probes; and
the processing circuitry is configured to
provisionally determine a scheduled inspection time of each of the plurality of ultrasonic probes as the first inspection schedule from the inspection history information including inspection history of the plurality of ultrasonic probes,
adjust the first inspection schedule in such a manner that, for ultrasonic probes of a same type, usage time indicated in the usage schedule and an inspection time indicated in the first inspection schedule do not overlap with each other, and
determine the second inspection schedule of the plurality of ultrasonic probes in accordance with the adjusted first inspection schedule.

6. The schedule management apparatus according to claim 1, wherein:
the at least one ultrasonic probe comprises a plurality of probes; and
the processing circuitry is configured to
provisionally determine a scheduled inspection time of each of the plurality of ultrasonic probes as the first inspection schedule from the inspection history information including inspection history of the plurality of ultrasonic probes,
adjust the first inspection schedule in such a manner that (i) for ultrasonic probes of a same type, usage time indicated in the usage schedule and inspection time indicated in the first inspection schedule do not overlap with each other, and (ii) inspection times for ultrasonic probes of a similar type do not overlap each other, and
determine the second inspection schedule of the plurality of ultrasonic probes in accordance with the adjusted first inspection schedule.

7. The schedule management apparatus according to claim 1, wherein:
the at least one ultrasonic probe comprises a plurality of probes; and
the processing circuitry is configured to
provisionally determine a scheduled inspection time of each of the plurality of ultrasonic probes as the first inspection schedule from the inspection history information including inspection history of the plurality of ultrasonic probes,
adjust the first inspection schedule in such a manner that (i) for ultrasonic probes of a same type, usage time indicated in the usage schedule and inspection time indicated in the first inspection schedule do not overlap with each other, and (ii) when there are a plurality of probes that do not overlap in both of the usage time and the inspection time, a probe having closer usage time is inspected earlier, and
determine the second inspection schedule of the plurality of ultrasonic probes in accordance with the adjusted first inspection schedule.

8. The schedule management apparatus according to claim 1, wherein the processing circuitry is configured to update the second inspection schedule each time the usage schedule of the ultrasonic probe is changed along with change in the examination reservation information.

9. The schedule management apparatus according to claim 1, wherein the processing circuitry is configured to acquire the inspection history information of the ultrasonic probe from a plurality of ultrasonic diagnostic apparatuses connected via a network.

10. An ultrasonic diagnostic apparatus comprising an ultrasonic probe and processing circuitry, the processing circuitry being configured to:
provisionally determine a first inspection schedule of the ultrasonic probe from inspection history information of the ultrasonic probe;
acquire examination reservation information of an object;
presume a usage schedule of the ultrasonic probe from the examination reservation information; and
determine a second inspection schedule of the ultrasonic probe based on the first inspection schedule and the usage schedule, by adjusting the first inspection schedule in such a manner that usage time of the ultrasonic probe in the usage schedule does not overlap with inspection time of the ultrasonic probe in the first inspection schedule.

11. A non-transitory computer-readable storage medium storing a schedule management program for causing a computer to execute processing comprising:
provisionally determining a first inspection schedule of the ultrasonic probe from inspection history information of the ultrasonic probe;
acquiring examination reservation information of an object;
presuming a usage schedule of the ultrasonic probe from the examination reservation information; and
determining a second inspection schedule of the ultrasonic probe based on the first inspection schedule and the usage schedule, by adjusting the first inspection schedule in such a manner that usage time of the ultrasonic probe in the usage schedule does not overlap with inspection time of the ultrasonic probe in the first inspection schedule.
